(19) United States
(12) Patent Application Publication
(10) Pub. No.: US 2026/0158238 A1
(43) Pub. Date: Jun. 11, 2026

(54) STEERABLE UNEVEN DOUBLE LUMEN CANNULA

(71) Applicant: Boston Scientific Medical Device Limited, Galway (IE)

(72) Inventors: Neeraj Garg, Gurgaon (IN); Swami Upadhyay, Raipur (IN); Takahiro Kobayashi, Nishitokyo-shi (JP); Kensuke Hayashi, Penang (MY); Mamoru Takenaka, Osaka (JP)

(73) Assignee: Boston Scientific Medical Device Limited, Galway (IE)

(21) Appl. No.: 19/413,419

(22) Filed: Dec. 9, 2025

Related U.S. Application Data
(60) Provisional application No. 63/730,635, filed on Dec. 11, 2024.

(51) Int. Cl. A61M 25/00; A61M 25/01; A61M 25/06

(52) U.S. Cl. CPC A61M 25/0026; A61M 25/0136; A61M 25/0147; A61M 25/0662; A61M 2025/0037; A61M 2025/015; A61M 2210/1057

(57) ABSTRACT

A steerable uneven double lumen cannula includes an elongate tube having proximal and distal ends, first and second lumens of different diameters, and a steering wire lumen. A wire filament may extend through the steering wire lumen. The wire filament may exit and reenter the steering wire lumen through a plurality of openings in the outer surface of the elongate tube. A handle may be coupled to the proximal end and may be configured to apply tension to the wire filament to control steering of the distal end.

### CROSS-REFERENCE TO RELATED APPLICATIONS

This application claims the benefit of U.S. Provisional Patent Application Ser. No. 63/730,635 filed on Dec. 11, 2024, the disclosure of which is incorporated herein by reference.

### FIELD

The disclosure pertains to medical devices, and methods for manufacturing and use of these medical devices. More particularly, the present disclosure pertains to medical devices for accessing a body lumen along a biliary and/or pancreatic tract.

### BACKGROUND

Endoscopic retrograde cholangiopancreatography (ERCP) is a medical procedure that combines upper gastrointestinal endoscopy and x-ray imaging to diagnose and treat conditions affecting the bile ducts and the pancreatic ducts. The procedure utilizes specialized endoscopic equipment, such as a duodenoscope, which is inserted through the patient's mouth and guided through the esophagus and stomach to the duodenum. During ERCP procedures, physicians navigate the scope while maintaining control of its proximal end for various functions including irrigation, visualization, insufflation, and device management. The procedure enables the exchange of specialized devices, such as ERCP cannulas, through a biopsy port for both diagnostic and therapeutic interventions. These cannulas facilitate access to the pancreaticobiliary system, allowing for guidewire placement, contrast dye injection, and various therapeutic maneuvers under x-ray guidance. There is an ongoing need to provide alternative cannulation devices for accessing ducts, such as, but not limited to, the common bile duct and the pancreatic duct as well as alternative methods for manufacturing and using medical devices.

### SUMMARY

This disclosure provides design, material, manufacturing method, and use alternatives for medical devices and medical systems.

In a first example, a steerable medical device may comprise an elongate tube having a proximal end and a distal end, a first lumen having a first diameter, a second lumen having a second diameter less than the first diameter, a steering wire lumen, a wire filament extending through the steering wire lumen, a plurality of openings extending from an outer surface of the elongate tube to the steering wire lumen, wherein the wire filament exits and reenters the steering wire lumen through the plurality of openings, and a handle coupled to the proximal end of the elongate tube and configured to apply tension to the wire filament.

Alternatively or additionally to any of the examples above, in another example, the plurality of openings may be axially spaced and circumferentially aligned.

Alternatively or additionally to any of the examples above, in another example, the wire filament may comprise 304V stainless steel spring temper wire.

Alternatively or additionally to any of the examples above, in another example, a distal end of the wire filament may be secured within a hypotube.

Alternatively or additionally to any of the examples above, in another example, the hypotube may be crimped at one or more axial locations to secure the wire filament to the hypotube.

Alternatively or additionally to any of the examples above, in another example, the handle may comprise a body portion and an actuating portion movably coupled to the body portion.

Alternatively or additionally to any of the examples above, in another example, the actuating portion may be configured to move axially relative to the body portion to apply and release tension to the wire filament.

Alternatively or additionally to any of the examples above, in another example, a distal opening of first lumen may be proximal to a distal opening of the second lumen.

Alternatively or additionally to any of the examples above, in another example, the handle may comprise an actuating member rotatably coupled to a body portion at a pinion point.

Alternatively or additionally to any of the examples above, in another example, rotation of the actuating member in a first direction may apply tension to the wire filament.

Alternatively or additionally to any of the examples above, in another example, rotation of the actuating member in a second direction opposite the first direction may release tension from the wire filament.

Alternatively or additionally to any of the examples above, in another example, the handle may include a ring member extending from a proximal end thereof configured to receive a thumb or finger of a user.

Alternatively or additionally to any of the examples above, in another example, the plurality of openings may comprise two or more openings.

Alternatively or additionally to any of the examples above, in another example, the steerable medical device may further comprise adhesive securing the hypotube within the steering wire lumen.

Alternatively or additionally to any of the examples above, in another example, the plurality of openings may each comprise beveled edges.

In another example, a steerable medical device may comprise an elongate tube having a proximal end and a distal end, a first guidewire lumen having a first diameter, a second guidewire lumen having a second diameter smaller than the first diameter, a first steering wire lumen, a second steering wire lumen, a first wire filament extending through the first steering wire lumen, a second wire filament extending through the second steering wire lumen, two or more openings extending from an outer surface of the elongate tube to the first steering wire lumen, wherein the first wire filament exits and reenters the first steering wire lumen through the two or more openings, two or more openings extending from the outer surface of the elongate tube to the second steering wire lumen, wherein the second wire filament exits and reenters the second steering wire lumen through the two or more openings, and a handle comprising an actuation member rotatably coupled to a body portion and connected to the first and second wire filaments.

Alternatively or additionally to any of the examples above, in another example, rotation of the actuation member in a first direction may apply tension to the first wire filament and release tension from the second wire filament.

Alternatively or additionally to any of the examples above, in another example, rotation of the actuation member in a second direction opposite the first direction may apply tension to the second wire filament and release tension from the first wire filament.

Alternatively or additionally to any of the examples above, in another example, the first and second wire filaments may comprise 304V stainless steel spring temper wire.

Alternatively or additionally to any of the examples above, in another example, distal ends of the first and second wire filaments may be secured within a respective first hypotube and second hypotube.

Alternatively or additionally to any of the examples above, in another example, the first hypotube may be crimped at one or more locations to secure the first wire filament thereto and the second hypotube may be crimped at one or more locations to secure the second wire filament thereto.

Alternatively or additionally to any of the examples above, in another example, the first diameter may be 0.035 inches and the second diameter may be 0.025 inches.

Alternatively or additionally to any of the examples above, in another example, the first wire filament may be releasably secured to the actuation member with a first connection member and the second wire filament may be releasably secured to the actuation member with a second connection member.

Alternatively or additionally to any of the examples above, in another example, the first connection member may be radially aligned with the second connection member.

In another example, a steerable medical device may comprise an elongate tube having a proximal end and a distal end, a first lumen having a first diameter, a second lumen having a second diameter smaller than the first diameter, a steering wire lumen, a wire filament extending through the steering wire lumen, a plurality of openings extending from an outer surface of the elongate tube to the steering wire lumen, wherein the plurality of openings define pivot points, and a handle coupled to the proximal end of the elongate tube and configured to apply tension to the wire filament.

Alternatively or additionally to any of the examples above, in another example, the wire filament may comprise 304V stainless steel spring temper wire.

Alternatively or additionally to any of the examples above, in another example, the handle may comprise a body portion and an actuating portion movably coupled to the body portion.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various configurations and together with the description serve to explain the principles of the present disclosure.

FIG. 1 is a schematic view of an illustrative system for accessing an opening of a body lumen of a subject;

FIG. 2 is a schematic view of the system depicted in FIG. 1, taken from circle-2;

FIG. 3 is a schematic view of an illustrative system for accessing a body lumen;

FIG. 4 is a schematic cross-sectional view of the illustrative elongate tube, taken at line 4-4 of FIG. 3;

FIG. 5 is a schematic perspective view of the distal end region of the elongate tube of FIG. 3;

FIG. 6 is a schematic cross-sectional view of the distal end region of the elongate tube of FIG. 3;

FIG. 7 is a schematic perspective view of the distal end region of the wire filament of FIG. 5 inserted into a hypotube;

FIG. 8 is a schematic perspective view of the illustrative handle that may be used to actuate the wire filament of FIG. 5 to deflect the distal tip of the elongate tube;

FIG. 9 is a schematic cross-sectional view of the illustrative handle of FIG. 8;

FIG. 10 is a schematic perspective view of the distal end region of the elongate tube having an alternative wire filament arrangement;

FIG. 11 is a schematic perspective view of the distal end region of the elongate tube having another alternative wire filament arrangement;

FIG. 12 is a schematic side view of another illustrative accessory medical device; and

FIG. 13 is a schematic cross-sectional view of the illustrative elongate tube, taken at line 13-13 of FIG. 12.

While the disclosure is amenable to various modifications and alternative forms, specifics thereof have been shown by way of example in the drawings and will be described in detail. It should be understood, however, that the intention is not to limit the invention to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

Although configurations of the present disclosure may be described with specific reference to medical devices and systems (e.g., endoscopic devices, accessory tools, and/or guidewires inserted through a duodenoscope, near or through a papilla, or the like) for selective access to, aligning with, and/or cannulation of the common bile duct (CBD) or pancreatic duct (PD) during an Endoscopic Retrograde Cholangiopancreatography (ERCP) procedure, it should be appreciated that such medical devices and systems may be used in a variety of medical procedures which require navigating one or more accessory tools through ductal, luminal, vascular, or body lumen anatomies, including, for example, interventional radiology procedures, balloon angioplasty/angiography procedures, thrombolysis procedures, urological or gynecological procedures, and the like. The disclosed medical devices and systems may be inserted via different access points and approaches, e.g., percutaneously, endoscopically, laparoscopically, or some combination thereof.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed configuration(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, it would be within the knowledge of one skilled in the art to effect the particular feature, structure, or characteristic in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some configurations, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

As used in this disclosure, the terms “upper” and “lower” are relative terms used to differentiate between opposite directions, not the overall orientation of the device. One of ordinary skill will recognize that a device could operate, for example, with the “uppermost” component at the bottom and the “lowermost” component at the top (i.e., “upside-down” when comparing the device operation to the terms), or in any other absolute orientation relative to the user and/or gravity.

The detailed description is intended to illustrate but not limit the disclosure. Those skilled in the art will recognize that the various elements described may be arranged in various combinations and configurations without departing from the scope of the disclosure. The detailed description illustrates example configurations of the disclosure.

In some instances, the devices and methods that are disclosed herein may be useful for diagnostic or therapeutic procedures in the biliary and/or pancreatic tracts, along with being useful for other purposes. Access to the pancreaticobiliary system, as facilitated by the devices disclosed herein, may be required to diagnose and/or treat a variety of conditions, including but not limited to tumors, gallstones, infections, sclerosis, and pseudo cysts. The devices disclosed herein may also be useful for navigation in other parts of the body such as the cardiovascular system and so forth.

Endoscopic retrograde cholangiopancreatography (ERCP) may be used to diagnose and treat conditions of the common bile duct, including, for example, gallstones, inflammatory strictures, leaks (e.g., from trauma, surgery, etc.), and cancer. In an ERCP procedures, a physician may view, through an endoscope, the inside of the stomach and/or the duodenum. Often, dyes may be injected (e.g., via a lumen of a sphincterotome or other device) into the ducts in the biliary tree and pancreas so that the area can be seen using X-rays. These procedures may necessitate gaining and keeping access to the papilla of Vater, the common bile duct, and/or the pancreatic duct, which may be technically challenging, may require extensive training and practice to gain proficiency, and may require one or more expensive tools in order to perform.

During an ERCP procedure, a number of steps are typically performed while the patient is often sedated and/or anaesthetized. For example, an endoscope or duodenoscope may be inserted through the mouth, down the esophagus, into the stomach, through the pylorus into the duodenum, to a position at or near the papilla of Vater (also referred to as the ampulla of Vater), which is the opening of the common bile duct and the pancreatic duct. Due to the shape of the papilla, and the angle at which the common bile and pancreatic ducts meet the wall of the duodenum, the distal end of the endoscope or duodenoscope is generally placed just past the papilla. Due to this positioning beyond the papilla, the endoscopes or duodenoscopes typically used in these procedures are usually side-viewing devices. The side-viewing feature provides imaging along the lateral aspect of the tip rather than from the end of the endoscope. Such orientation may allow a clinician to obtain an image of the medial wall of the duodenum, where the papilla of Vater is located, even though the distal tip of the viewing device is beyond the opening. Once the papilla or a target area in the duodenum is visually located, an accessory medical device, such as sphincterotome, cannula, or other accessory medical device, may be extended out a side opening or window of the endoscope or duodenoscope for facilitating access through the papilla and into a desired one of the common bile duct and the pancreatic duct.

Although not required, a fluid (e.g., a compressible fluid, such as compressible gas, air, nitrogen, carbon dioxide, etc., and/or other suitable fluid) may be delivered via a catheter (e.g., as inserted through a subject as discussed above or otherwise inserted), a sphincterotome, a cannula, and/or other elongated tubular device extending through an inserted endoscope, and/or other suitable elongate medical device to an opening (e.g., the papilla of Vater) of a body lumen of a subject. Applying the fluid to the opening may facilitate identifying and/or accessing the body lumen by at least partially dilating the opening with the fluid prior to and/or during delivery of a catheter, sphincterotome, guidewire, etc. to a target anatomical structure or tissue (e.g., a duct or other suitable structure or tissue) at or distal of the opening of the body lumen. Some example techniques and systems for cannulating an opening of a body lumen using fluid are described in U.S. Patent Application Publication No. 2019/0380565, which was filed on Jun. 13, 2019, and titled DEVICES, SYSTEMS AND METHOD FOR ACCESSING A BODY LUMEN, which is hereby incorporated by reference for any and all purposes.

Alternatively or in addition to using fluid to open the opening of the body lumen, the opening may be manually opened by engaging a distal end or tip of an accessory medical device with the papilla. Further, the accessory medical device may be manipulated and/or the endoscope through which the accessory medical device extends may be manipulated to direct the accessory medical device through the papilla and into a desired one of the common bile duct and the pancreatic duct. Some example techniques and systems for manipulating a distal tip of a medical device, such as a guidewire, to access a desired body lumen are described in U.S. Patent Application Publication No. 2015/0073391, which was filed on Sep. 11, 2014, and titled MEDICAL DEVICE WITH A MOVABLE TIP, which is hereby incorporated by reference for any and all purposes.

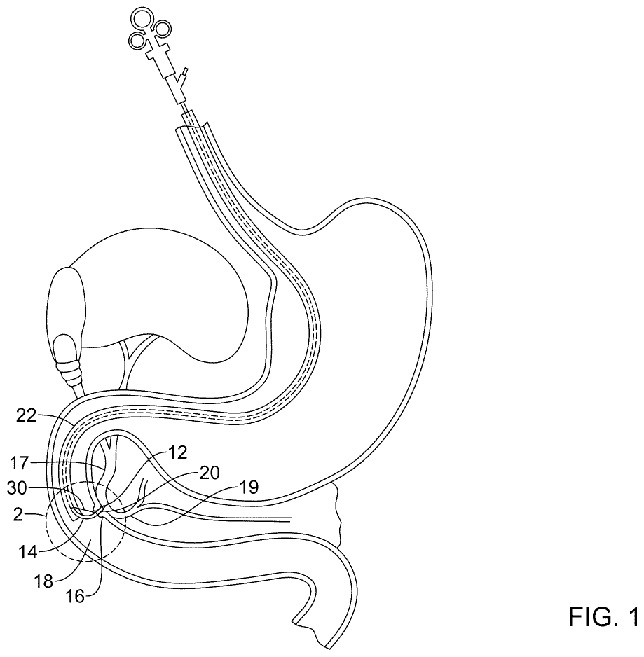

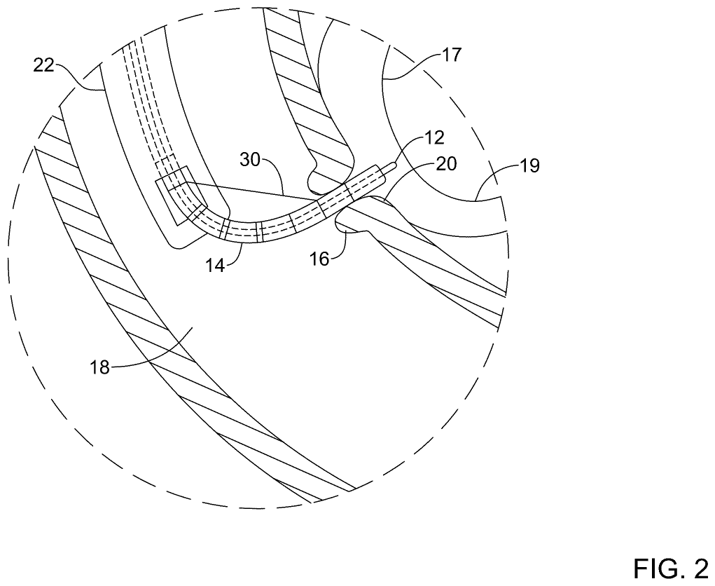

Turning to the Figures, FIGS. 1 and 2 depict a selective cannulation during an ERCP procedure, which includes a guidewire 12 and/or an accessory medical device 14 (e.g., an endoscopic accessory device, such as a cannula, a sphincterotome, and/or other suitable accessory medical device) being passed towards, against, and/or through a body lumen such as the major papilla 16 (e.g., ampullary entry) near the descending duodenum 18 to access the Sphincter of Oddi Complex 20. During the cannulation procedure, a distal portion of a medical device 22 (e.g., an endoscope, duodenoscope, guide catheter, etc.) may be positioned within the descending duodenum 18. The guidewire 12 and the accessory medical device 14 may be advanced through a working channel (e.g., a lumen) of the medical device 22 towards the major papilla 16. Additionally, the guidewire 12 and/or the accessory medical device 14 may be advanced against and/or through the major papilla 16 to one of the common bile duct 17 and the pancreatic duct 19.

Accessing the papilla 16, the common bile duct 17, and/or the pancreatic duct 19 may be difficult because the openings are small compared to a size (e.g., a diameter or other size measurement) of many accessory medical devices, the openings may be closely located, the openings may be completely collapsed/closed, the openings may extend into the descending duodenum 18 at an angle that may be difficult to visualize and/or access, and/or it may be difficult to control a position of a distal tip of the accessory medical device 14. Thus, a medical professional may be required to manipulate the accessory medical device 14 and guidewire 12 by manually rotating the medical device 22, pulling on a wire filament 30 to adjust the accessory medical device 14 in a single direction, and/or use an elevator and/or other suitable ramped surface within the distal end of the medical device 22 in an attempt to align or orient the accessory medical device 14 and/or the guidewire 12 with respect to the medical device 22 and the openings of the papilla 16, the common bile duct 17, and/or the pancreatic duct 19. Difficult cannulation procedures in which the medical professional fails to access the body lumen within a certain time limit or after a certain number of unsuccessful attempts may lead to significant post-procedure complications, such as post-ERCP pancreatitis (PEP).

A medical professional accessing a body lumen (e.g., a duct, a papilla, a common bile duct, a pancreatic duct, or the like) by manipulating an accessory medical device against or into the opening of the body lumen, as discussed above, may subject the walls of the opening of the body lumen to compressive forces. Compression of a body lumen opening or the body lumen itself can cause buckling in what may be described as the “accordion effect”. As such, it is desirable to have control over a distal tip of the accessory medical device 14 such that a user may precisely access an opening of the papilla 16 and once through the papilla 16, direct the accessory medical device 14 and/or guidewire 12 to a desired one of the common bile duct 17 and the pancreatic duct 19.

In some cases, it may be difficult to cannulate the biliary tree. In such cases, alternative methods of cannulating the biliary tree may be used. In one example, a second guidewire may be used to assist in cannulation. For example, one guidewire may be dedicated to the pancreatic duct and another dedicated for biliary cannulation. The guidewire in the pancreatic duct may be used to straighten the duct and adjust to the axis of the guidewire in the direction of the bile duct while attempting to insert the second guidewire. However, this method of cannulation may include several challenges, such as, but not limited to, multiple attempts to access the papilla with the second guidewire for adjustment purposes, due to the need for repeated guidewire manipulations and adjustments, the procedure can be lengthy, multiple access attempts at the papilla can potentially cause damage to the papillary tissue, and the introduction of contrast dye and guidewire into the pancreatic duct during this method may lead to complications such as pancreatitis.

In other examples, an uneven double lumen cannula (UDLC) may be used. The UDLC method may be a specialized cannulation technique that utilizes a double lumen catheter with distinct lumen diameters including, for example, 0.025 inches for a distal lumen and 0.035 inches for a proximal lumen. The uneven positioning of these lumens creates a specialized channel within the tip. The UDLC may be intubated to the papilla through the use of a pancreatic guidewire using the distal lumen. This action may straighten both the pancreatic duct and the common channel. Biliary cannulation may then be performed using the proximal lumen. The UDLC may allow for simultaneous injection and suction while using the guidewire as well as enable guidewire replacement or exchange between 0.025″ and 0.035″ sizes. In some cases, the UDLC method may provide more effective papilla stabilization compared to the two-guidewire method described above. Further, the UDLC method may be monitored through both endoscopic imaging and fluoroscopic x-ray. Further, the guidewire extending from the proximal lumen may be observed during biliary cannulation. However, a UDLC may not allow for the manipulation of the distal end of the UDLC device to adjust the direction for accessing the common bile duct and the pancreatic duct. Thus, it may take a long time to cannulate the bile duct and/or cause damage to the papilla. Disclosed herein are UDLC devices which include actuation mechanisms to facilitate adjustment of the distal end of UDLC device.

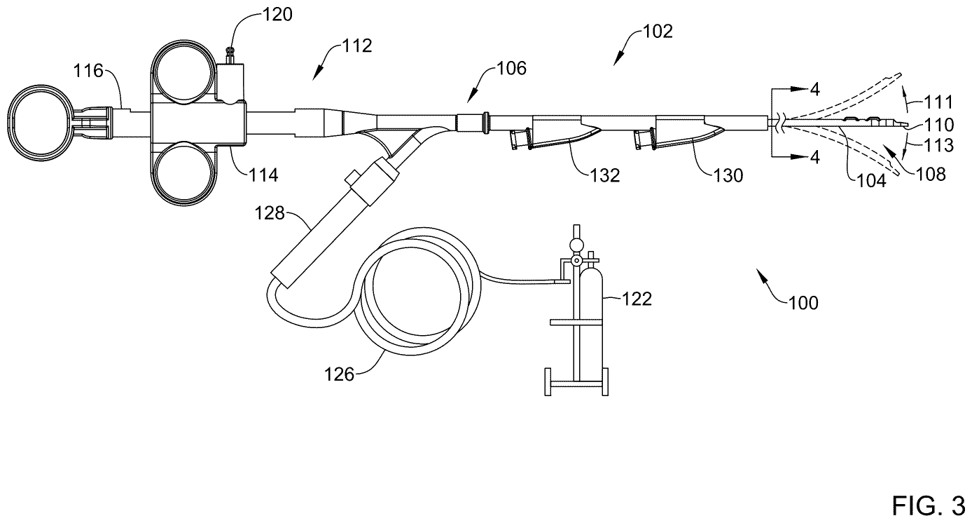

FIG. 3 schematically depicts an illustrative system 100 for accessing a body lumen. In some cases, the system 100 may include, among other components, an accessory medical device 102, similar in form and function to the accessory medical device 14, with a flexible elongate shaft or tube 104. The tube 104 may have a proximal end region 106 (disposed within a handle 112) and a distal end region 108. The distal end region 108 of the tube 104 may have a distal tip 110 configured to be directed toward and/or through an opening of a body lumen. The proximal end region 106 of the tube 104 may be connected to, coupled with, or otherwise in communication with a handle 112.

The handle 112 may be any suitable type of handle. In one example, the handle 112 may have a first portion 114 configured to receive fingers (e.g., receive two fingers) of a user using the accessory medical device 102 to hold the accessory medical device 102 and a second portion 116 configured to receive a thumb of the user. In some cases, the first portion 114 of the handle 112 may be an actuating portion of the handle 112 and a user may adjust the first portion 114 relative to the second portion 116 to control positioning of a distal end of the accessory medical device 102, as discussed herein or otherwise. As schematically depicted in FIG. 3, the first portion 114 of the handle 112 may have two finger holes configured to receive two fingers from the user and the second portion 116 may have a thumb hole configured to receive a thumb from the user, but other configurations of the first and second portions 114, 116 of the handle 112 are contemplated. Although not required, the handle 112, as illustrated, may be held and/or operated with a single hand of a user.

The first portion 114 of the handle 112 may be configured to be adjusted or manipulated to control a position of a distal end region 108 of the elongate tube 104. In some cases, the first portion 114 may be configured to adjust axially or longitudinally to adjust a position of the distal end region 108 of the elongate tube 104 in a plurality of directions. Alternatively, or additionally, in some cases, the first portion 114 may be configured to adjust radially or rotationally to adjust a position of the distal end region 108 of the elongate tube 104 in a plurality of directions

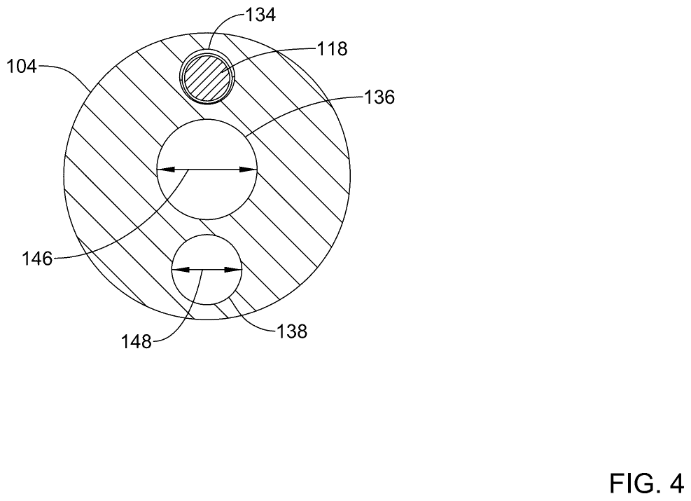

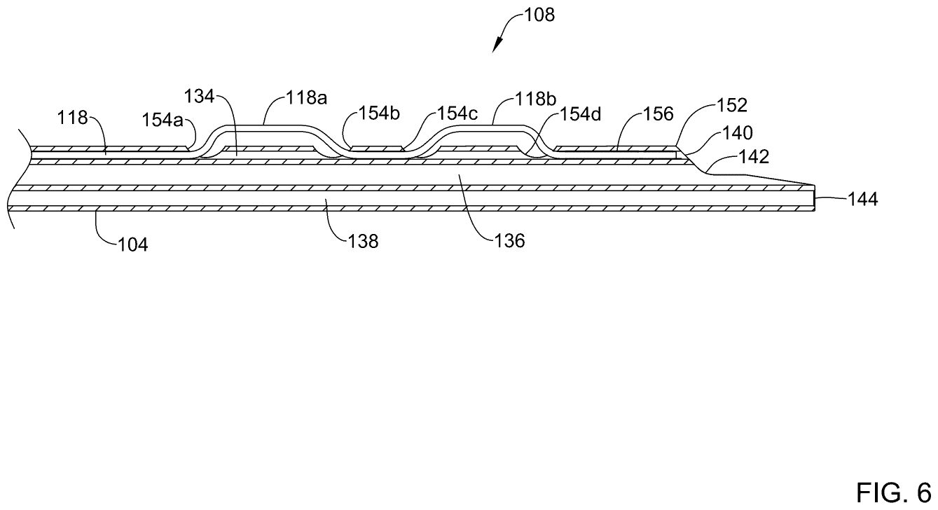

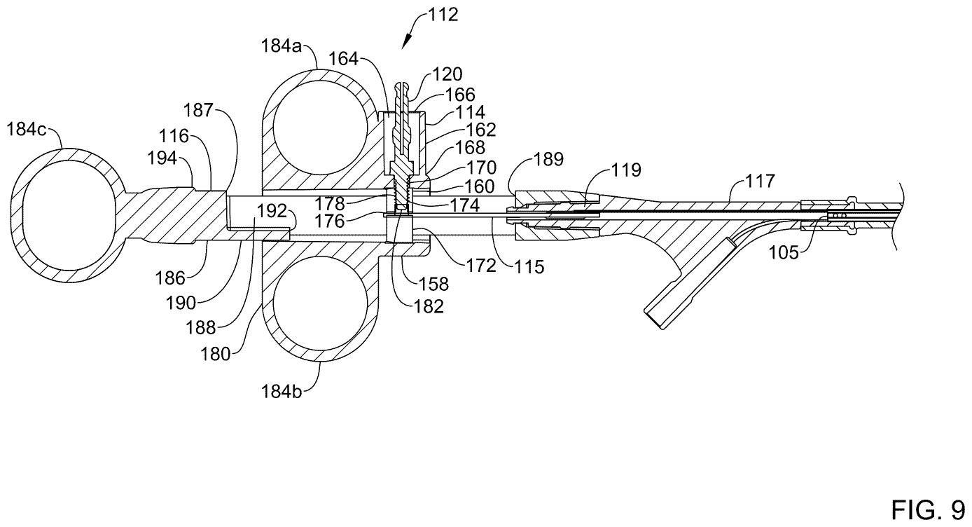

A wire filament 118 (see, for example, FIG. 4) may extend along the elongate tube 104 and may be actuated to control or steer the distal end region 108 of the elongate tube 104. In some cases, the wire filament 118 may be a steering wire. The wire filament 118 may have a proximal end region 180 (see, for example, FIG. 9) connected to the first portion 114 of the handle 112, and a proximal portion extending at least partially within the tube 104. The wire filament 118 also may have a distal end 152 (see, for example, FIG. 6) connected to the distal end region 108 of the elongate tube 104. The wire filament 118 may be a 304V stainless steel spring temper wire. However, other materials may be used, as desired.

The wire filament 118 may be received within a lumen 134 (see, for example, FIG. 4) of the tube 104 at least along a proximal portion of the filament 118 and may be configured to slide within the lumen 134. In some cases, the first portion 114 of the handle 112 may be coupled to the wire filament 118 and manipulated to slide at least a portion of the wire filament 118 along the tube 104 (e.g., within the lumen of the tube 104). In one example, the first portion 114 of the handle 112 may be longitudinally adjusted relative to the second portion 116 of the handle 112 to apply and remove tension to and/or from the wire filament 118.

Sliding or adjusting the wire filament 118 may result in the distal end region 108 of the elongate tube 104 moving responsively to the sliding of the wire filament 118. As such, the first portion 114 of the handle 112 may be manipulated to control or adjust a position of the distal end region 108 of the elongate tube 104, for example, to direct the distal end region 108 toward the opening of a body lumen. In some cases, natural tension in or acting on the tube 104 and/or the wire filament 118 may result in the wire filament 118 returning to a relaxed position or state once a tension applied to the wire filament 118 via the handle 112 is released.

In some cases, the wire filament 118 may be utilized to electrically cut or remove tissue of the subject (e.g., to cut and cauterize tissue) and may be considered a cautery wire. As such, the handle 112 or other suitable portion of the medical device 22 may include an electrical connection at a connection member 120 for an energy source (e.g., a radiofrequency energy source, or the like) to energize the wire filament 118 and facilitate cutting tissue. However, this is not required. Alternatively, or additionally the connection member 120 may be releasably secured to the wire filament 118.

The distal tip 110 of the elongate tube 104 may be flexible and configured to adjust in response to steering with or adjustment of a control configuration at a proximal end of the accessory medical device 102. To facilitate precisely controlling a distal end of the accessory medical device 102 while the device 102 is within a subject, the distal tip 110 may be configured to be adjustable or steerable in two or more directions through control at the proximal end of the accessory medical device 102 such that distal end region 108 of the elongate tube 104 may be guided to a desired body lumen. The distal tip 110 may be a steerable distal tip.

The distal tip 110 may be formed from any suitable material configured to bend, deflect, or otherwise move in response to forces acting thereon. In one example, the distal tip may be formed from a polyether block amide (PEBA, such as, for example, PEBAX®), but this is not required, and other suitable materials are contemplated.

A positioning of the distal tip 110 may be controlled or adjusted in any suitable manner. In one example, the wire filament 118 may be actuated to control a position of the distal tip 110. As will be described in more detail herein, a distal end of the wire filament 118 may be coupled to the distal tip 110 and/or a proximal end of the wire filament 118 may be coupled to the first portion 114 of the handle 112 in any suitable manner. In one example, the wire filament 118 may be coupled to the distal tip 110 and/or the first portion 114 of the handle 112 using adhesion, for example, through ultraviolet (UV) curing techniques, crimping techniques, mechanical fixation techniques, and/or other suitable connecting or coupling techniques.

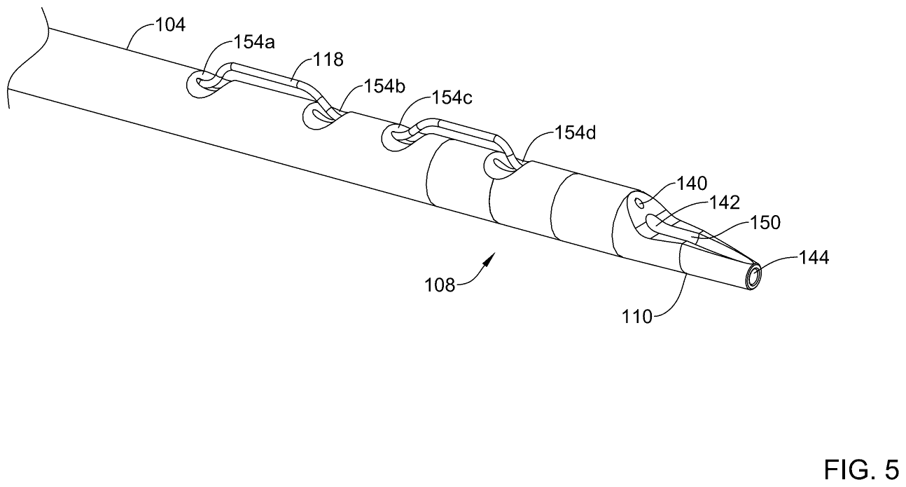

Referring additionally to FIG. 4, which is a cross-sectional view of the illustrative elongate tube 104, taken at line 4-4 of FIG. 3, in addition to the one or more lumens 134 in which the wire filament 118 for controlling a position of the distal tip 110 extends, the accessory medical device 102 may include one or more other lumens extending from at least the proximal end region 106 of the tube 104 to the distal end region 108 of the tube 104. For example, the elongate tube 104 may include a first guidewire lumen 136 and a second guidewire lumen 138. In some cases, one or more of the lumens 134, 136, 138 may each have a distal opening 140, 142, 144 (see, for example, FIG. 5) through the distal tip 110 of the elongate tube 104. In some cases, a first guidewire or other suitable medical devices may extend through the first guidewire lumen 136 of the elongate tube 104 and proximally out of a first aperture port 130. In some cases, a second guidewire or other suitable medical devices may extend through the second guidewire lumen 138 of the elongate tube 104 and proximally out of a second aperture port 132. The aperture ports 130, 132 may be located at the distal end of the handle 112 and/or at one or more other suitable locations. In some cases, the aperture ports 130, 132 and the tube 104 may be configured so that one or more of the guidewires and/or other suitable medical devices may be stripped through the side of the tube 104 for a rapid removal or exchange of devices, but this is not required.

The first guidewire lumen 136 may be generally centrally located or extend co-linearly with a longitudinal axis of the elongate tube 104. The steering wire lumen 134 may be radially offset from the longitudinal axis of the elongate tube 104 in a first direction. The second guidewire lumen 138 may be radially offset from the longitudinal axis of the elongate tube 104 in a second direction, opposite the first direction. Said differently, the steering wire lumen 134 and the second guidewire lumen 138 may each extend along the elongate tube 104 at a radial location closer to an outer surface of the elongate tube 104 than the first guidewire lumen 136. However, the lumens 134, 136, 138 may be arranged or positioned in different configurations, as desired.

The first guidewire lumen 136 may have a first diameter, or cross-sectional dimension, 146 and the second guidewire lumen 138 may have a second diameter, or cross-sectional dimension, 148. In some examples, the first diameter 146 may be greater than the second diameter 148. It is contemplated that the first guidewire lumen 136 may be sized and shaped to receive a 0.035-inch (0.889 millimeters) guidewire while the second lumen 138 may be sized and shaped to receive a 0.025-inch (0.635 millimeters) guidewire. The reverse configuration is also contemplated in which the second lumen 138 is larger than the first lumen 136. The distal opening 142 of the first guidewire lumen 136 may be proximal to the distal opening 144 of the second lumen 138. For example, the distal tip 110 may taper or reduce in outer profile to define a laterally accessible channel 150 extending from the distal opening 142 of the first guidewire lumen 136 to the distal end of the elongate tube 104.

In some cases, a lumen of the one or more lumens of the elongate tube 104 may have a fluid connection at a proximal end for a fluid source (e.g., a pressurized fluid source 122 such as a CO₂ tank, a contrast fluid source such as a syringe, etc.) The lumen may be configured to apply a fluid from the fluid source through an opening at the distal end region 108 of the elongate tube 104 to a body lumen of the subject

Although FIG. 3 depicts the fluid source 122 coupled to a fluid port 124 of the accessory medical device 102 via a fluid tube 126 and a flow regulator 128, the fluid source 122 and/or other suitable fluid sources (e.g., syringes, etc.) may be coupled to the fluid port 124 in other suitable manners. In one example, a syringe with contrast fluid may be coupled to the fluid port 124 via a luer lock connection and/or other suitable connection. Contrast agent may be delivered into the body lumen and may be used to temporarily improve imaging of the inside of the body lumen by, for example, x-ray, computed tomography (CT), or magnetic resonance (MR) imaging, ultrasound, and the like. Alternatively, separate lumens and separate fluid ports may be provided for each of a pressurized fluid and a contrast fluid. In some cases, the fluid source 122 may be omitted.

FIG. 5 is a perspective view of the distal end region 108 of the elongate tube 104. FIG. 6 is a cross-sectional view of the distal end region 108 of the elongate tube 104. The wire filament 118 may extend distally from the handle 112, through the lumen 134 to a distal end 152 disposed proximal to the distal opening 140 of the steering wire lumen 134. In some cases, the elongate tube 104 may include one or more openings 154a, 154b, 154c, 154d extending from an outer surface of the elongate tube 104 to steering wire lumen 134. In some configurations, the one or more openings 154a-d may have angled or beveled edges. For example, the edges of the openings 154a-d may extend at a generally non-orthogonal angle to the longitudinal axis of the elongate tube 104. In some cases, the angled or beveled edges may help guide the wire filament 118 in and out of steering wire lumen 134. However, this is not required. The one or more openings 154a-d may be axially spaced and circumferentially aligned.

The one or more openings 154a-d may create lateral access locations to the steering wire lumen 134. The openings 154a-d may stop or end before reaching the first guidewire lumen 136. Said differently, the first guidewire lumen 136 may be fluidly isolated from the steering wire lumen 134 adjacent to the one or more openings 154a-d. In some examples, the wire filament 118 may enter and/or exit the steering wire lumen 134 through the one or more openings 154a-d such that one or more regions of the wire filament 118 extend along an exterior surface of the elongate tube 104. Said differently, the wire filament 118 may weave in and out of the steering wire lumen 134 through the one or more openings 154a-d. In the illustrated configuration, the wire filament 118 includes a first region 118a and a second region 118b extending external to the lumen 134 or along an exterior surface of the elongate tube 104. However, fewer than two or more than two regions of the wire filament 118 may be external to the steering wire lumen 134. Further, the elongate tube 104 may include fewer than four or more than four openings 154a-d, as desired. The openings 154a-d may be uniformly spaced or eccentrically spaced as desired. For example, there may be a same or uniform distance between adjacent openings 154a-d or there may be at least some differing distances between adjacent openings 154a-d. It is contemplated that the placement of the openings 154a-d may be varied to allow for customization of the steering characteristics.

It is contemplated that having the distal end region of the wire filament 118 enter and exit the steering wire lumen 134 may allow facilitate distribution of steering forces along the distal end region 108 of the elongate tube 104. In some cases, the one or more openings 154a-d and/or the weaving arrangement of the wire filament 118 may provide more precise control over the bending movements by creating specific pivot points at the entry and/or exit locations of the wire filament 118. It is further contemplated that partially exposing the wire filament 118 exterior to the elongate tube 104 may allow the wire filament 118 to maintain internal support while providing external steering capabilities.

To assemble the wire filament 118 with the elongate tube 104, the distal end 152 of the wire filament 118 may be inserted into the proximal opening (not explicitly shown) of the steering wire lumen 134 of the elongate tube 104. The wire filament 118 may be distally advanced through the steering wire lumen 134 until the distal end 152 of the wire filament 118 reaches the proximal most opening 154a. The elongate tube 104 may then be gently bent to guide the wire filament 118 out of the steering wire lumen 134 through the proximal most opening 154a. The distal end 152 of the wire filament 118 may then be reinserted into the steering wire lumen 134 through the next opening 154b. This may be repeated for the next two openings. For example, the elongate tube 104 may be gently bent to guide the wire filament 118 out of the steering wire lumen 134 through the third opening 154c and the wire filament 118 may be guided back into the steering wire lumen 134 through the fourth or distal most opening 154d. If the elongate tube 104 is provided with more than four openings 154a-d, the wire filament 118 may be guided out of the steering wire lumen 134 and back into the lumen 134, as needed.

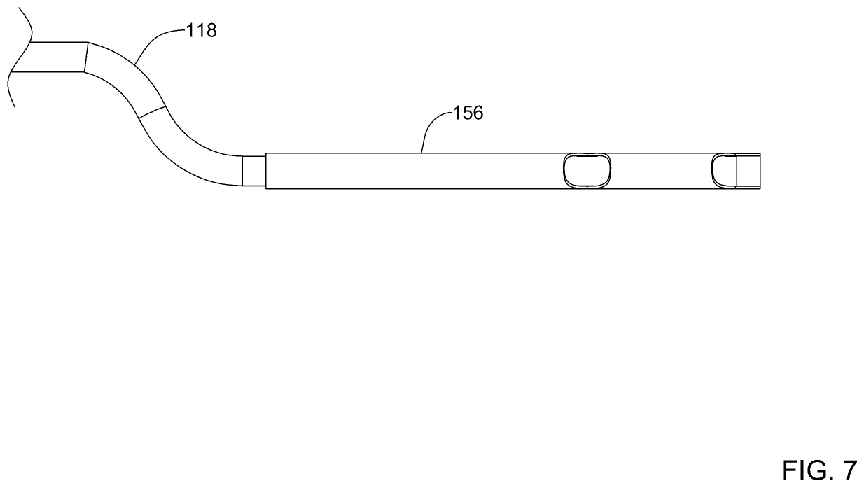

After reinserting the wire filament 118 back into the steering wire lumen 134 through the distal most opening 154d, the wire filament 118 may be distally advanced such that the distal end 152 of the wire filament 118 extends distally from the distal opening 140 of the steering wire lumen 134. The distal end region of the wire filament 118 may be inserted into the lumen of a hypotube 156. FIG. 7 is a perspective view of the distal end region of the wire filament 118 inserted into a hypotube 156. The hypotube 156 and the wire filament 118 may be crimped at one or more axial locations. For example, a radially inward force may be applied to an outer surface of the hypotube 156 to mechanically deform the hypotube 156 and/or the wire filament 118 to secure the hypotube 156 to the wire filament 118. Next, glue or adhesive may be applied to the outer surface of the hypotube 156. The wire filament 118 and hypotube 156 assembly may then be proximally retracted (e.g., by applying a proximal pulling force to the proximal end of the wire filament 118) back into the steering wire lumen 134. The proximal retracting force may be applied until the hypotube 156 is in the desired axial location. For example, the hypotube 156 may be positioned between a distal end of the distal most opening 154d and the distal opening 140 of the steering wire lumen 134. However, the hypotube 156 may be positioned at other axial locations, as desired. The glue/adhesive may secure the distal end region of the wire filament 118 within the steering wire lumen 134 such that the distal end 152 of the wire filament 118 is held in a fixed axial location relative to distal tip 110 of the elongate tube 104.

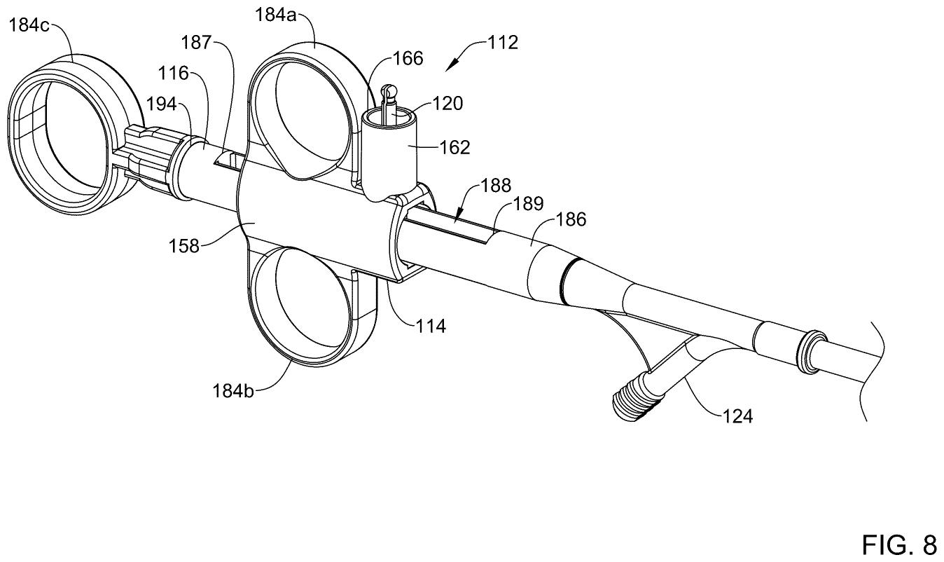

The wire filament 118 may be actuated at its proximal end to selectively deflect the distal end region 108 of the elongate tube 104. FIG. 8 is a perspective view of the illustrative handle 112 that may be used to actuate the wire filament 118 to deflect the distal tip 110 of the elongate tube 104. FIG. 9 is a cross-sectional view of the illustrative handle 112. In FIGS. 8 and 9, the handle 112 is in a neutral configuration in which the distal tip 110 of the elongate tube 104 extends colinearly with the proximal portion of the elongate tube 104 (absent an external biasing force from another device). The first portion 114 of the handle 112 may be axially displaceable relative to the second portion 116 of the handle 112. For example, the first portion 114 may be proximally retracted relative to the second portion 116 to deflect the distal tip 110 of the elongate tube 104 in a first direction and the first portion 114 may be distally advanced relative to the second portion 116 to deflect the distal tip 110 in a second direction opposite the first direction. For example, proximal retraction of the first portion 114 may move the distal tip 110 in a direction towards the wire filament 118, as shown at arrow 111 in FIG. 3 and distal advancement of the first portion 114 may move the distal tip 110 in a direction away from the wire filament 118, as shown at arrow 113 in FIG. 3. Said differently, proximal retraction of the first portion 114 may bend the distal tip 110 such that the wire filament 118 is within an interior of the curved portion and distal advancement of the first portion 114 may bend the distal tip 110 such that the wire filament 118 extends along an exterior or outer region of the curved portion.

The first portion 114 may include a generally tubular main body portion 158 defining a lumen 160 extending from proximal end to a distal end of the tubular main body portion 158. The lumen 160 may extend generally coaxially with a proximal end region of the elongate tube 104. The first portion 114 may further include a generally tubular branch member 162 extending generally orthogonal to the main body portion 158. The branch member 162 may define a lumen 164 extending from a laterally outward end 166 to a laterally inward end 168 fixedly coupled to the body portion. In some configurations, the main body portion 158 and the branch member 162 may be formed as a single monolithic structure. However, this is not required.

The lumen 164 of the branch member 162 may be configured to receive the connection member 120 therethrough. The connection member 120 may include a threaded region 170 configured to threadably engage a mating threaded region 174 of a mounting member 172. The mounting member 172 may be slidably disposed within the lumen 160 of the main body portion 158. The mounting member 172 may include a first aperture 176 extending from a proximal end to a distal end of the mounting member 172. The first aperture 176 may be configured to receive a proximal end region 180 of the wire filament 118 therethrough. The mounting member 172 may further include a second aperture 178 extending generally orthogonal to the first aperture 176 and parallel to the lumen 164 of the branch member 162. The threaded region 174 of the mounting member 172 may be formed in the luminal wall of the second aperture 178. The second aperture 178 may be connected to the first aperture 176 to allow a radially inward portion 182 of the connection member 120 to contact the wire filament 118. For example, once the wire filament 118 is secured to the distal end region 108 of the elongate tube 104, the proximal end region 180 of the wire filament 118 may be inserted into the first aperture 176 of the mounting member 172. The connection member 120 may then be rotated to move radially inwards to secure the proximal end region 180 of the wire filament 118 between the connection member 120 and the mounting member 172. As the proximal end region 180 of the wire filament 118 is secured to the first portion 114 of the handle 112 via the mounting member 172 and the connection member 120, movement of the first portion 114 may be translated to the wire filament 118.

In some configurations, the wire filament 118 may be formed from two or more wire elements fixedly secured to one another. For example, the wire filament 118 may include a proximal wire member 115 and a distal wire member 117. The distal wire member 117 may be fixedly secured to an outer surface of the proximal wire member 115 at a connection point 119 using coupling techniques, such as, but not limited to, welding, soldering, brazing, adhesives, crimping, brackets, other mechanical coupling means, or the like. Forming the wire filament 118 from two or more components may allow the proximal wire member 115 to extend generally co-linearly with the longitudinal axis of the proximal portion of the elongate tube 104 and the distal wire member 117 to be radially offset from the longitudinal axis of the proximal portion of the elongate tube 104. However, the wire filament 118 may be formed as a single monolithic member, if so desired.

The first portion 114 of the handle 112 may include a first ring member 184a and a second ring member 184b extending laterally from the main body portion 158 and each configured to receive a finger. The clinician may pull proximally on the ring members 184a, 184b to actuate the distal tip 110 in a first direction or push distally on the ring members 184a, 184b to actuation the distal tip 110 in a second direction, opposite the first direction.

The second portion 116 of the handle 112 may include a generally cylindrical body portion 186 including a ring member 184c extending from a proximal end thereof. The ring member 184c may be configured to receive a thumb or finger of the clinician to hold the second portion 116 relative to the first portion 114 of the handle 112.

The body portion 186 may include a slot 188 extending through a thickness or across a diameter thereof. The slot 188 may extend from a proximal end 187 to a distal end 189. The mounting member 172 of the first portion 114 of the handle 112 may be axially displaceable along the slot 188. In some configurations, the slot 188 may not extend across an entirety of the diameter of the body portion 186 for an entire length of the slot 188. For example, the slot 188 may extend through less than an entirety of the diameter of the body portion 186 along a proximal portion 190 of the slot 188 to define a mechanical stop 192. Said differently, slot 188 may be configured to create a mechanical stop 192 along a region thereof to limit movement of the first portion 114 of the handle 112. For example, as the first portion 114 is proximally retracted a proximal end of the mounting member 172 may contact the mechanical stop 192 to prevent further proximal movement of the first portion 114. It is contemplated that the length of the slot 188 which does not extend across the entire diameter of the body portion 186 may be selected based on the desired proximal movement of the first portion 114 (and the desired amount of bend at the distal tip 110). In other examples, the length of proximal retraction of the first portion 114 of the handle 112 may be limited by mechanical engagement of the proximal end of the main body portion 158 of the first portion 114 engaging a transition region 194 of the second portion 116. The transition region 194 may be a change in diameter of the second portion 116 from a first diameter to a second, larger, diameter. In some cases, the diameter of the body portion 186 may change. In other examples, the transition region 194 may be a part of the ring member 184c.

It is further contemplated that a mechanical stop may be provided to limit distal movement of the first portion 114 of the handle 112. In some cases, the mechanical stop may be the distal end 189 of the slot 188. For example, the distal end of the mounting member 172 may contact the distal end 189 of the slot 188 to limit distal movement of the first portion 114 of the handle 112. In other configurations, the slot 188 may extend through less than an entirety of the diameter of the body portion 186 (similar to portion 190) to define a mechanical stop.

The first portion 114 of the handle 112 may be assembled with the second portion 116 such that the body portion 186 of the second portion 116 is disposed within the lumen 160 of the main body portion 158 of the first portion 114. The first portion 114 may be axially displaceable along an outer surface of the second portion 116. The mounting member 172, the connection member 120 and the wire filament 118 may extend into the slot 188 of the second portion 116.

The fluid port 124 may be coupled with the second portion 116 of the handle 112 using known techniques, such as, but not limited to, press-fits, friction fits, over-molding, heat-shrinking, or the like. The proximal end 105 of the elongate tube 104 may be coupled with the fluid port 124 using known techniques, such as, but not limited to, press-fits, friction fits, over-molding, heat-shrinking, mechanical coupling members such as pins, or the like. Further, the first and second aperture ports 130, 132 may be coupled with the elongate tube 104 using known techniques, such as, but not limited to, over-molding, heat-shrinking, or the like. The elongate tube 104 may include side ports adjacent to the first and second aperture ports 130, 132 to allow a guidewire to enter the respective guidewire lumen 136, 138 from the aperture port 130, 132.

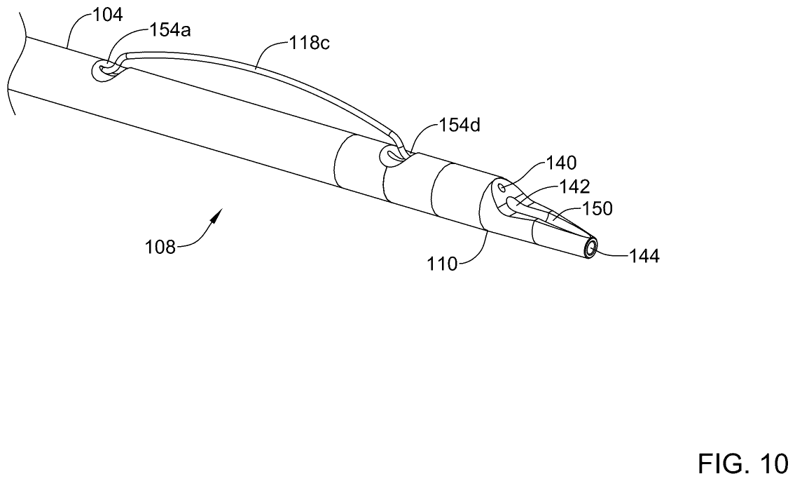

FIG. 10 is a perspective view of the distal end region 108 of the elongate tube 104 having an alternative wire filament 118 arrangement. In FIG. 10, the elongate tube 104 includes a proximal opening 154a and distal opening 154d to provide lateral access to the steering wire lumen 134. The intermediate openings 154b, 154c of FIG. 5 have been omitted. The proximal and distal openings 154a, 154d extend from an outer surface of the elongate tube 104 to steering wire lumen 134. In some configurations, the openings 154a, 154d may have angled or beveled edges. For example, the edges of the openings 154a, 154d may extend at a generally non-orthogonal angle to the longitudinal axis of the elongate tube 104. In some cases, the angled or beveled edges may help guide the wire filament 118 in and out of steering wire lumen 134. However, this is not required. The proximal and distal opening 154a, 154d may be axially spaced and circumferentially aligned.

Similar to FIG. 5, the proximal and distal openings 154a, 154d may create lateral access locations to the steering wire lumen 134. The openings 154a, 154d may stop or end before reaching the first guidewire lumen 136. Said differently, the first guidewire lumen 136 may be fluidly isolated from the steering wire lumen 134 adjacent to the openings 154a, 154d. In some examples, the wire filament 118 may enter and/or exit the steering wire lumen 134 through the one or more openings 154a, 154d such that at least one region of the wire filament 118 extends along an exterior surface of the elongate tube 104. Said differently, the wire filament 118 may weave in and out of the steering wire lumen 134 through the proximal and distal openings 154a, 154d. In the illustrated configuration, the wire filament 118 includes a first or single region 118c extending external to the lumen 134 or along an exterior surface of the elongate tube 104. However, fewer than one or more than one region of the wire filament 118 may be external to the steering wire lumen 134. Further, the elongate tube 104 may include fewer than two or more than two openings 154a, 154d, as desired.

It is contemplated that having the distal end region of the wire filament 118 enter and exit the steering wire lumen 134 may facilitate distribution of steering forces along the distal end region 108 of the elongate tube 104. In some cases, the proximal and distal openings 154a, 154d and/or the weaving arrangement of the wire filament 118 may provide more precise control over the bending movements by creating specific pivot points at the entry and/or exit locations of the wire filament 118. It is further contemplated that partially exposing the wire filament 118 exterior to the elongate tube 104 may allow the wire filament 118 to maintain internal support while providing external steering capabilities. In some cases, having a continuous length of wire filament 118 extending external to the elongate tube 104 (e.g., having a similar length as the distance between the proximal and distal openings 154a, 154d of FIGS. 5 and 6) may provide direct force transmission to the distal tip 110 which may increase the response of the distal tip 110 to actuation of the first portion 114 of the handle 112.

The configuration illustrated in FIG. 10 may have a simplified routing of the wire filament 118 relative to the configuration of FIGS. 5 and 6. For example, only two openings 154a, 154d may be required. Further, the wire filament 118 may exit and re-enter the steering lumen 134 once which may simplify assembly of the wire filament 118 with the elongate tube 104. For example, the wire filament 118 may be assembled with the elongate tube 104 in a similar manner as described above with respect to FIGS. 5 and 6. The wire filament 118 may be crimped to a hypotube and the hypotube adhesively secured to the elongate tube 104.

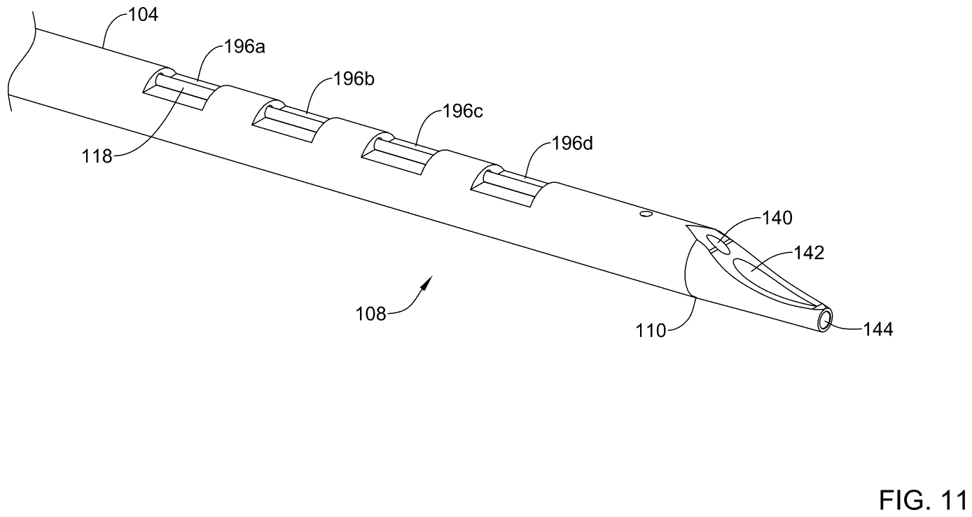

FIG. 11 is a perspective view of the distal end region 108 of the elongate tube 104 having an alternative wire filament 118 arrangement. In FIG. 11, the elongate tube 104 includes a proximal opening 196a, a first intermediate opening 196b, a second intermediate opening 196c, and a distal opening 196d to provide lateral access to the steering wire lumen 134. The openings 196a-d may extend from an outer surface of the elongate tube 104 to steering wire lumen 134. In some configurations, the openings 196a-d may have rectangular cuts that are free from angled or beveled edges. For example, the edges of the openings 196a-d may extend at a generally orthogonal angle to the longitudinal axis of the elongate tube 104. However, this is not required. The openings 196a-d may be axially spaced and circumferentially aligned.

Similar to FIG. 5, the openings 196a-d may create lateral access locations to the steering wire lumen 134. The openings 196a-d may stop or end before reaching the first guidewire lumen 136. Said differently, the first guidewire lumen 136 may be fluidly isolated from the steering wire lumen 134 adjacent to the openings 196a-d. In some examples, the wire filament 118 may remain within the steering wire lumen 134 adjacent to the openings. While the wire filament 118 remains within the steering wire lumen 134, the openings 196a-d may create pivot points to enable more precise control over the bending movements. The elongate tube 104 may include fewer than four or more than four openings 196a-d, as desired. The openings 196a-d may be uniformly spaced or eccentrically spaced as desired. For example, there may be a same or uniform distance between adjacent openings 196a-d or there may be at least some differing distances between adjacent openings 196a-d. It is contemplated that the placement of the openings 196a-d may be varied to allow for customization of the steering characteristics.

The configuration illustrated in FIG. 11 may have a simplified routing of the wire filament 118 relative to the configuration of FIGS. 5 and 6. For example, fully the wire filament 118 may be fully contained within the steering wire lumen 134 for a length of the wire filament 118 while maintaining steering capabilities. This may simplify assembly and maintain a cleaner external profile of the elongate tube 104. The wire filament 118 may be assembled with the elongate tube 104 in a similar manner as described above with respect to FIGS. 5 and 6. The wire filament 118 may be crimped to a hypotube and the hypotube adhesively secured to the elongate tube 104.

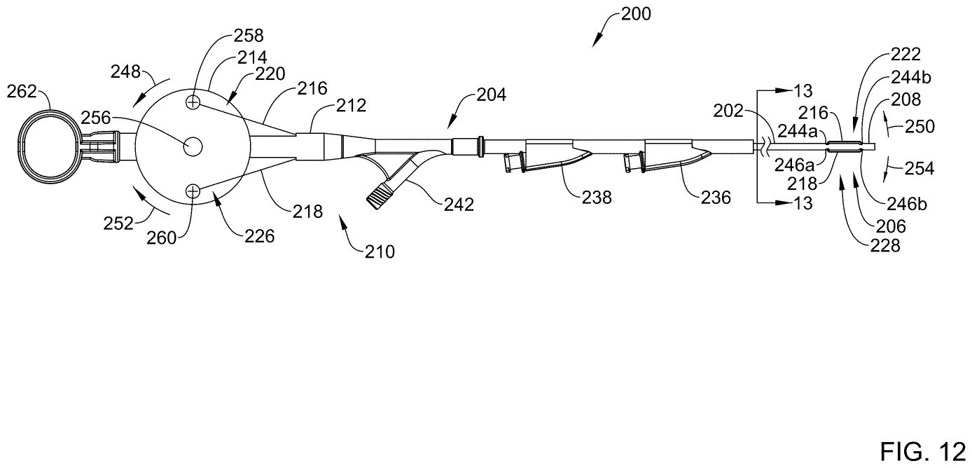

FIG. 12 schematically depicts another illustrative accessory medical device 200, similar in form and function to accessory medical device 14, that may be used with a system to access a lumen, such as, but not limited to system 100 or medical device 22. The accessory medical device 200 may include a flexible elongate shaft or tube 202. The tube 202 may have a proximal end region 204 (disposed within a handle 210) and a distal end region 206. The distal end region 206 of the tube 202 may have a distal tip 208 configured to be directed toward and/or through an opening of a body lumen. The proximal end region 204 of the tube 202 may be connected to, coupled with, or otherwise in communication with a handle 210.

The handle 210 may be any suitable type of handle. In one example, the handle 210 may have a body portion 212 configured to receive a thumb of a user using the accessory medical device 200 to hold the accessory medical device 200 and an actuation member or actuation portion 214 configured to be actuated to control positioning of a distal end of the accessory medical device 200, as discussed herein or otherwise. Although not required, the handle 210, as illustrated, may be held and/or operated with a single hand of a user.

The actuation member 214 of the handle 210 may be configured to be adjusted or manipulated to control a position of a distal end region 206 of the elongate tube 202. In some cases, the actuation member 214 may be configured to be rotated to adjust a position of the distal end region 206 of the elongate tube 202 in a plurality of directions. Alternatively, or additionally, in some cases, the actuation member 214 may be configured to adjust axially to adjust a position of the distal end region 206 of the elongate tube 202 in a plurality of directions

A first wire filament 216 and a second wire filament 218 may extend along the elongate tube 202 and may be actuated to control or steer the distal end region 206 of the elongate tube 202. In some cases, the first and second wire filaments 216, 218 may be steering wires. The first wire filament 216 may have a proximal end region 220 connected to the actuation member 214 of the handle 210, and a proximal portion extending at least partially within the tube 202. The first wire filament 216 also may have a distal end (not explicitly shown) connected to the distal end region 206 of the elongate tube 202. The second wire filament 218 may have a proximal end region 226 connected to the actuation member 214 of the handle 210, and a proximal portion extending at least partially within the tube 202. The second wire filament 218 also may have a distal end (not explicitly shown) connected to the distal end region 206 of the elongate tube 202. The wire filaments 216, 218 may be 304V stainless steel spring temper wires. However, other materials may be used, as desired.

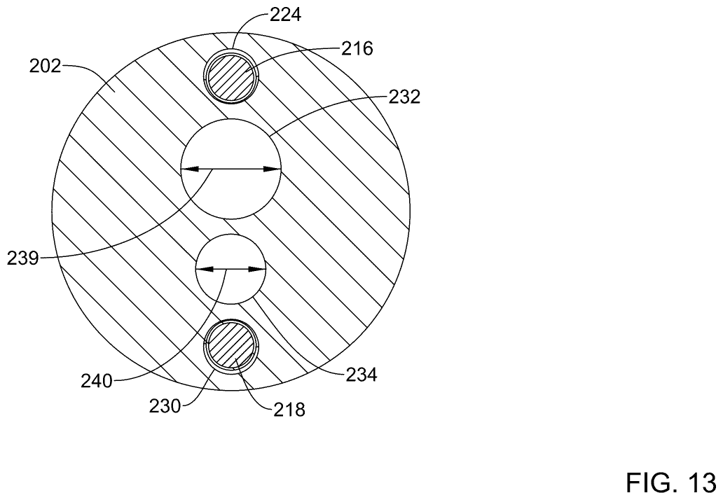

The first wire filament 216 may be received within a lumen 224 (see, for example, FIG. 13) of the tube 202 at least along a proximal portion of the filament 216 and may be configured to slide within the lumen 224. The lumen 224 may be a first steering wire lumen. In some cases, the actuation member 214 of the handle 210 may be coupled to the wire filament 216 and manipulated to slide at least a portion of the wire filament 216 along the tube 202 (e.g., within the lumen of the tube 202). In one example, the body portion 212 of the handle 210 may be rotated relative to the body portion 212 of the handle 210 to apply and remove tension to and/or from the first wire filament 216.

The second wire filament 218 may be received within a lumen 230 (see, for example, FIG. 13) of the tube 202 at least along a proximal portion of the filament 218 and may be configured to slide within the lumen 230. The lumen 230 may be a second steering wire lumen. In some cases, the actuation member 214 of the handle 210 may be coupled to the wire filament 216 and manipulated to slide at least a portion of the wire filament 218 along the tube 202 (e.g., within the lumen of the tube 202). In one example, the body portion 212 of the handle 210 may be rotated relative to the body portion 212 of the handle 210 to apply and remove tension to and/or from the second wire filament 218.

Sliding or adjusting the wire filaments 216, 218 may result in the distal end region 206 of the elongate tube 202 moving responsively to the sliding of the wire filaments 216, 218. As such, the actuation member 214 of the handle 210 may be manipulated to control or adjust a position of the distal end region 206 of the elongate tube 202, for example, to direct the distal end region 206 toward the opening of a body lumen. In some cases, natural tension in or acting on the tube 202 and/or the wire filaments 216, 218 may result in the wire filament 216 returning to a relaxed position or state once a tension applied to the wire filaments 216, 218 via the handle 210 is released.

In some cases, one or both of the wire filaments 216, 218 may be utilized to electrically cut or remove tissue of the subject (e.g., to cut and cauterize tissue) and may be considered a cautery wire. As such, the handle 210 or other suitable portion of the medical device 22 may include an electrical connection (not explicitly shown) for an energy source (e.g., a radiofrequency energy source, or the like) to energize the wire filament(s) 216, 218 and facilitate cutting tissue. However, this is not required.

The distal tip 208 of the elongate tube 202 may be flexible and configured to adjust in response to steering with or adjustment of a control configuration at a proximal end of the accessory medical device 200. To facilitate precisely controlling a distal end of the accessory medical device 200 while the device 200 is within a subject, the distal tip 208 may be configured to be adjustable or steerable in two or more directions through control at the proximal end of the accessory medical device 200 such that distal end region 206 of the elongate tube 202 may be guided to a desired body lumen. The distal tip 208 may be a steerable distal tip.

The distal tip 208 may be formed from any suitable material configured to bend, deflect, or otherwise move in response to forces acting thereon. In one example, the distal tip may be formed from a polyether block amide (PEBA, such as, for example, PEBAX®), but this is not required, and other suitable materials are contemplated.

A positioning of the distal tip 208 may be controlled or adjusted in any suitable manner. In one example, the wire filaments 216, 218 may be actuated to control a position of the distal tip 208. As will be described in more detail herein, a distal end of the wire filaments 216, 218 are each coupled to the distal tip 208 and/or a proximal end of the wire filaments 216, 218 are coupled to the actuation member 214 of the handle 210 in any suitable manner. In one example, the wire filaments 216, 218 may be coupled to the distal tip 208 and/or the actuation member 214 of the handle 210 using adhesion, for example, through ultraviolet (UV) curing techniques, crimping techniques, mechanical fixation techniques, and/or other suitable connecting or coupling techniques.

Referring additionally to FIG. 13, which is a cross-sectional view of the illustrative elongate tube 202, taken at line 13-13 of FIG. 12, in addition to the one or more lumens 224, 230 in which the wire filaments 216, 218 for controlling a position of the distal tip 208 extend, the accessory medical device 200 may include one or more other lumens extending from at least the proximal end region 204 of the tube 202 to the distal end region 206 of the tube 202. For example, the elongate tube 202 may include a first guidewire lumen 232 and a second guidewire lumen 234. In some cases, one or more of the lumens 224, 230, 232, 234 may each have a distal opening (not explicitly shown) through the distal tip 208 of the elongate tube 202. In some configurations, the distal openings may be arranged in a similar manner to the distal openings 140, 142, 144 described with respect to FIG. 5. However, this is not required. In some configurations, the distal end region 206 of the elongate tube 202 may be free from a taper or have a substantially uniform cross-sectional dimension along an entirety of a length thereof. In such a configuration, each of the distal openings of the lumens 224, 230, 232, 234 may be at a distal end of the elongate tube 202.

In some cases, a first guidewire or other suitable medical devices may extend through the first guidewire lumen 232 of the elongate tube 202 and proximally out of a first aperture port 236. In some cases, a second guidewire or other suitable medical devices may extend through the second guidewire lumen 234 of the elongate tube 202 and proximally out of a second aperture port 238. The aperture ports 236, 238 may be located at the distal end of the handle 210 and/or at one or more other suitable locations. In some cases, the aperture ports 236, 238 and the tube 202 may be configured so that one or more of the guidewires and/or other suitable medical devices may be stripped through the side of the tube 202 for a rapid removal or exchange of devices, but this is not required.

The lumens 224, 230, 232, 234 may be radially aligned across a diameter (or cross-sectional dimension) of the elongate tube 202. In some cases, the steering wire lumens 224, 230 may be positioned at the most radially outward positions at opposing sides of the diameter. This may allow the steering wires 216, 218 to exit the respective steering wire lumen 224, 230 and extend exterior to the elongate tube 202 along a portion of the length of the steering wires 216, 218, as will be described in more detail herein. The first guidewire lumen 232 may be disposed radially inwards of the first steering wire lumen 224. The second guidewire lumen 234 may be disposed radially inwards of the second steering wire lumen 230. Each of the lumens 224, 230, 232, 234 may extend generally parallel to a longitudinal axis of the elongate tube 202. However, the lumens 224, 230, 232, 234 may be arranged or positioned in different configurations, as desired.

The first guidewire lumen 232 may have a first diameter, or cross-sectional dimension, 239 and the second guidewire lumen 234 may have a second diameter, or cross-sectional dimension, 240. In some examples, the first diameter 239 may be greater than the second diameter 240. It is contemplated that the first guidewire lumen 232 may be sized and shaped to receive a 0.035-inch (0.889 millimeters) guidewire while the second lumen 234 may be sized and shaped to receive a 0.025-inch (0.635 millimeters) guidewire. The reverse configuration is also contemplated in which the second lumen 234 is larger than the first lumen 232.

While not explicitly shown, in some configurations, the distal opening of the first guidewire lumen 232 may be proximal to the distal opening of the second lumen 234. For example, the distal tip 208 may taper or reduce in outer profile to define a laterally accessible channel extending from the distal opening of the first guidewire lumen 232 to the distal end of the elongate tube 202. In other configurations, each of the first and second guidewire lumens 232, 234 may have distal openings at similar longitudinal locations.

In some cases, a lumen of the one or more lumens of the elongate tube 202 may have a fluid connection at a proximal end for a fluid source (e.g., a pressurized fluid source such as a CO₂ tank, a contrast fluid source such as a syringe, etc.) The lumen may be configured to apply a fluid from the fluid source through an opening at the distal end region 206 of the elongate tube 202 to a body lumen of the subject. For example, while not explicitly shown, a fluid source (similar to fluid source 122) may be coupled to a fluid port 242 in a similar manner to that described with respect to FIG. 3. In other examples, a syringe with contrast fluid may be coupled to the fluid port 242 via a luer lock connection and/or other suitable connection. Contrast agent may be delivered into the body lumen and may be used to temporarily improve imaging of the inside of the body lumen by, for example, x-ray, computed tomography (CT), or magnetic resonance (MR) imaging, ultrasound, and the like. Alternatively, separate lumens and separate fluid ports may be provided for each of a pressurized fluid and a contrast fluid. In some cases, the fluid source may be omitted.

The first wire filament 216 may extend distally from the handle 210, through the lumen 224 to a distal end disposed proximal to the distal end of the first steering wire lumen 224. In some cases, the elongate tube 202 may include one or more openings 244a, 244b extending from an outer surface of the elongate tube 202 to the first steering wire lumen 224. In some configurations, the one or more openings 244a, 224b may have angled or beveled edges. For example, the edges of the openings 244a, 244b may extend at a generally non-orthogonal angle to the longitudinal axis of the elongate tube 202. In some cases, the angled or beveled edges may help guide the first wire filament 216 in and out of steering wire lumen 224. However, this is not required. The one or more openings 244a, 244b may be axially spaced and circumferentially aligned.

The one or more openings 244a, 244b may create lateral access locations to the first steering wire lumen 224. The openings 244a, 244b may stop or end before reaching the first guidewire lumen 232. Said differently, the first guidewire lumen 232 may be fluidly isolated from the first steering wire lumen 224 adjacent to the one or more openings 244a, 244b. In some examples, the first wire filament 216 may enter and/or exit the first steering wire lumen 224 through the one or more openings 244a, 244b such that one or more regions 222 of the first wire filament 216 extend along an exterior surface of the elongate tube 202. Said differently, the first wire filament 216 may weave in and out of the steering wire lumen 224 through the one or more openings 244a, 244b. In the illustrated configuration, the first wire filament 216 includes a single region 222 extending external to the lumen 224 or along an exterior surface of the elongate tube 202. However, fewer than one or more than one region of the first wire filament 216 may be external to the steering wire lumen 224. Further, the elongate tube 202 may include fewer than two or more than two openings 244a, 244b, as desired. The openings 244a, 244b may be uniformly spaced or eccentrically spaced as desired. For example, there may be a same or uniform distance between adjacent openings 244a, 244b or there may be at least some differing distances between adjacent openings 244a, 244b. It is contemplated that the placement of the openings 244a, 244b may be varied to allow for customization of the steering characteristics.

It is contemplated that having the facilitate distribution of steering forces along the distal end region 206 of the elongate tube 202. In some cases, the one or more openings 244a, 244b and/or the weaving arrangement of the first wire filament 216 may provide more precise control over the bending movements by creating specific pivot points at the entry and/or exit locations of the first wire filament 216. It is further contemplated that partially exposing the first wire filament 216 exterior to the elongate tube 202 may allow the first wire filament 216 to maintain internal support while providing external steering capabilities.

The second wire filament 218 may extend distally from the handle 210, through the lumen 230 to a distal end disposed proximal to the distal end of the second steering wire lumen 230. In some cases, the elongate tube 202 may include one or more openings 246a, 246b extending from an outer surface of the elongate tube 202 to the second steering wire lumen 230. The one or more openings 246a, 246b of the second steering wire lumen 230 may be spaced circumferentially from the one or more openings 244a, 244b of the first steering wire lumen 224. For example, the one or more openings 246a, 246 b of the second steering wire lumen 230 may be about 180° from the one or more openings 244a, 244b of the first steering wire lumen 224. In some configurations, the one or more openings 246a, 224b may have angled or beveled edges. For example, the edges of the openings 246a, 246b may extend at a generally non-orthogonal angle to the longitudinal axis of the elongate tube 202. In some cases, the angled or beveled edges may help guide the second wire filament 218 in and out of steering wire lumen 230. However, this is not required. The one or more openings 246a, 246b may be axially spaced and circumferentially aligned.

The one or more openings 246a, 246b may create lateral access locations to the second steering wire lumen 230. The openings 246a, 246b may stop or end before reaching the second guidewire lumen 234. Said differently, the second guidewire lumen 234 may be fluidly isolated from the second steering wire lumen 230 adjacent to the one or more openings 246a, 246b. In some examples, the second wire filament 218 may enter and/or exit the second steering wire lumen 230 through the one or more openings 246a, 246b such that one or more regions 228 of the second wire filament 218 extend along an exterior surface of the elongate tube 202. Said differently, the second wire filament 218 may weave in and out of the steering wire lumen 230 through the one or more openings 246a, 246b. In the illustrated configuration, the second wire filament 218 includes a single region 228 extending external to the lumen 230 or along an exterior surface of the elongate tube 202. However, fewer than one or more than one region of the second wire filament 218 may be external to the steering wire lumen 230. Further, the elongate tube 202 may include fewer than two or more than two openings 246a, 246b, as desired. The openings 246a, 246b may be uniformly spaced or eccentrically spaced as desired. For example, there may be a same or uniform distance between adjacent openings 246a, 246b or there may be at least some differing distances between adjacent openings 246a, 246b. It is contemplated that the placement of the openings 246a, 246b may be varied to allow for customization of the steering characteristics. In some configurations, the proximal opening 246a of the second steering wire lumen 230 may be axially aligned with the proximal opening 244a of the first steering wire lumen 224. Similarly, the distal opening 246b of the second steering wire lumen 230 may be axially aligned with the distal opening 244b of the first steering wire lumen 224.

It is contemplated that having the distal end region of the second wire filament 218 enter and exit the steering wire lumen 230 may facilitate distribution of steering forces along the distal end region 206 of the elongate tube 202. In some cases, the one or more openings 246a, 246b and/or the weaving arrangement of the second wire filament 218 may provide more precise control over the bending movements by creating specific pivot points at the entry and/or exit locations of the second wire filament 218. It is further contemplated that partially exposing the second wire filament 218 exterior to the elongate tube 202 may allow the second wire filament 218 to maintain internal support while providing external steering capabilities.

To assemble the first wire filament 216 with the elongate tube 202, the distal end of the first wire filament 216 may inserted into the proximal opening (not explicitly shown) of the first steering wire lumen 224 of the elongate tube 202. The first wire filament 216 may be distally advanced through the first steering wire lumen 224 until the distal end of the first wire filament 216 reaches the proximal opening 244a. The elongate tube 202 may then be gently bent to guide the first wire filament 216 out of the first steering wire lumen 224 through the proximal opening 244a. The distal end of the first wire filament 216 may then be reinserted into the first steering wire lumen 224 through the distal opening 244b. This may be repeated, as needed, if there are more than two openings 244a, 244b.

After reinserting the first wire filament 216 back into the steering wire lumen 224 through the distal opening 244b, the first wire filament 216 may be distally advanced such that the distal end of the first wire filament 216 extends distally from the distal opening of the first steering wire lumen 224. The distal end region of the first wire filament 216 may be inserted into the lumen of a hypotube (not explicitly shown). The hypotube and the first wire filament 216 may be crimped at one or more axial locations. For example, a radially inward force may be applied to an outer surface of the hypotube to mechanically deform the hypotube and/or the first wire filament 216 to secure the hypotube to the first wire filament 216. Next, glue or adhesive may be applied to the outer surface of the hypotube. The first wire filament 216 and hypotube assembly may then be proximally retracted (e.g., by applying a proximal pulling force to the proximal end of the first wire filament 216) back into the first steering wire lumen 224. The proximal retracting force may be applied until the hypotube is in the desired axial location. For example, the hypotube may be positioned between a distal end of the distal opening 244b and the distal opening of the first steering wire lumen 224. However, the hypotube may be positioned at other axial locations, as desired. The glue/adhesive may secure the distal end region of the first wire filament 216 and they hypotube within the first steering wire lumen 224 such that the distal end of the first wire filament 216 is held in a fixed axial location relative to distal tip 208 of the elongate tube 202.

To assemble the second wire filament 218 with the elongate tube 202, the distal end of the second wire filament 218 may inserted into the proximal opening (not explicitly shown) of the second steering wire lumen 230 of the elongate tube 202. The second wire filament 218 may be distally advanced through the second steering wire lumen 230 until the distal end of the second wire filament 218 reaches the proximal opening 246a. The elongate tube 202 may then be gently bent to guide the second wire filament 218 out of the second steering wire lumen 230 through the proximal opening 246a. The distal end of the second wire filament 218 may then be reinserted into the second steering wire lumen 230 through the distal opening 246b. This may be repeated, as needed, if there are more than two openings 246a, 246b.

After reinserting the second wire filament 218 back into the steering wire lumen 230 through the distal opening 246b, the second wire filament 218 may be distally advanced such that the distal end of the second wire filament 218 extends distally from the distal opening of the second steering wire lumen 230. The distal end region of the second wire filament 218 may be inserted into the lumen of a hypotube (not explicitly shown). The hypotube and the second wire filament 218 may be crimped at one or more axial locations. For example, a radially inward force may be applied to an outer surface of the hypotube to mechanically deform the hypotube and/or the second wire filament 218 to secure the hypotube to the second wire filament 218. Next, glue or adhesive may be applied to the outer surface of the hypotube. The second wire filament 218 and hypotube assembly may then be proximally retracted (e.g., by applying a proximal pulling force to the proximal end of the second wire filament 218) back into the second steering wire lumen 230. The proximal retracting force may be applied until the hypotube is in the desired axial location. For example, the hypotube may be positioned between a distal end of the distal opening 246b and the distal opening of the second steering wire lumen 230. However, the hypotube may be positioned at other axial locations, as desired. The glue/adhesive may secure the distal end region of the second wire filament 218 and they hypotube within the second steering wire lumen 230 such that the distal end of the second wire filament 218 is held in a fixed axial location relative to distal tip 208 of the elongate tube 202.

The first wire filament 216 and the second wire filament 218 may each be actuated at its respective proximal end to selectively deflect the distal end region 206 of the elongate tube 202. In FIG. 12, the handle 210 is in a neutral configuration in which the distal tip 208 of the elongate tube 202 extends colinearly with the proximal portion of the elongate tube 202 (absent an external biasing force from another device). The actuation member 214 of the handle 210 may be rotationally displaceable relative to the body portion 212 of the handle 210. For example, the actuation member 214 may be rotated in a first direction relative to the body portion 212 to deflect the distal tip 208 of the elongate tube 202 in a first direction and the actuation member 214 may be rotated in a second direction opposite the first direction relative to the body portion 212 to deflect the distal tip 208 in a second direction opposite the first direction. For example, rotation of the actuation member 214 in a first direction (e.g., counterclockwise), as shown at arrow 248, of may move the distal tip 208 in a direction towards the first wire filament 216, as shown at arrow 250 in FIG. 12 and rotation of the actuation member 214 in a second direction (e.g., clockwise), as shown at arrow 252, may move the distal tip 208 in a direction towards the second wire filament 218, as shown at arrow 254 in FIG. 12. Said differently, rotation of the actuation member 214 in a first direction may bend the distal tip 208 such that the first wire filament 216 is within an interior of the curved portion and rotation of the actuation member 214 in a second direction may bend the distal tip 208 such that the first wire filament 216 extends along an exterior or outer region of the curved portion.

The actuation member 214 may be a wheel, knob, or other member rotationally coupled to the body portion 212. For example, the actuation member 214 may be movably secured relative to the body portion 212 at a pinion point 256. The pinion point 256 may be a pivot location between a first connection member 258 and a second connection member 260 that that connect to the first and second wire filaments 216, 218 to the actuation member 214, allowing the actuation member 214 to control bidirectional movement through clockwise and counterclockwise rotation. The pinion point 256 may be any suitable connection which allows for the actuation member 214 to rotate relative to the body portion 212. In some cases, the pinion point 256 may be a pin.

The first connection member 258 may removably couple the first wire filament 216 to the actuation member 214. In some examples, the first connection member 258 may be a screw or other mechanical coupling means configured to secure a proximal end of the first wire filament 216 within a first aperture of the actuation member 214. The second connection member 260 may removably couple the second wire filament 218 to the actuation member 214. In some examples, the second connection member 260 may be a screw or other mechanical coupling means configured to secure a proximal end of the second wire filament 218 within a second aperture of the actuation member 214. The first and second connection members 258, 260 may be axially aligned such that as the actuation member is rotated the first and second wire filaments 216, 218 are moved in opposing directions by an approximately equal distance. For example, when the actuation member 214 is rotated in a counterclockwise direction 248, the first wire filament 216 is pulled proximally to shorten a length of the first wire filament 216 (e.g., apply tension to the first wire filament 216) within the first steering wire lumen 224. Substantially simultaneously therewith, the second wire filament 218 is distally advanced to increase a length of the second wire filament 218 (e.g., release tension from the second wire filament 218) within the second steering wire lumen 230. This may cause the distal end region 206 to compress along the region adjacent to the first steering wire 216 and elongate along the region adjacent to the second steering wire 218 to bend the distal end region 206 in the first direction 250. Said differently, when the actuation member 214 is rotated in the counterclockwise direction, the first wire filament 216 may be disposed along the interior of the curved region. Further, when the actuation member 214 is rotated in a clockwise direction 252, the second wire filament 218 is pulled proximally to shorten a length of the second wire filament 218 (e.g., apply tension to the second wire filament 218) within the second steering wire lumen 230. Substantially simultaneously therewith, the first wire filament 216 is distally advanced to increase a length of the first wire filament 216 (e.g., release tension from the first wire filament 216) within the first steering wire lumen 224. This may cause the distal end region 206 to compress along the region adjacent to the second steering wire 218 and elongate along the region adjacent to the first steering wire 216 to bend the distal end region 206 in the second direction 254. Said differently, when the actuation member 214 is rotated in the clockwise direction, the second wire filament 218 may be disposed along the interior of the curved region.

The body portion 212 may include at least one aperture for rotatably receiving the pinion point 256 of the actuation member 24. Further, the body portion 212 may include one or more lumens for receiving a proximal portion of the first and second wire filaments 216, 218 therethrough. In some cases, separate lumens may be provided in the body portion 212 for each of the first and second wire filaments 216, 218. However, this is not required. The body portion 212 of the handle 210 may further include a ring member 262 extending from a proximal end thereof. The ring member 262 may be configured to receive a thumb or finger of the clinician to hold the body portion 212 relative to the actuation member 214 of the handle 210.

The fluid port 242 may be coupled with the body portion 212 of the handle 210 using known techniques, such as, but not limited to, press-fits, friction fits, over-molding, heat-shrinking, or the like. The proximal end of the elongate tube 202 may be coupled with the fluid port 242 using known techniques, such as, but not limited to, press-fits, friction fits, over-molding, heat-shrinking, mechanical coupling members such as pins, or the like. Further, the first and second aperture ports 236, 238 may be coupled with the elongate tube 202 using known techniques, such as, but not limited to, over-molding, heat-shrinking, or the like. The elongate tube 202 may include side ports adjacent to the first and second aperture ports 236, 238 to allow a guidewire to enter the respective guidewire lumen 232, 234 from the aperture port 236, 238.

The materials that can be used for the various components of the systems presently disclosed may include those commonly associated with medical devices. For simplicity purposes, the following discussion makes reference to system 100 and accessory devices 102, 200 referenced above. However, this is not intended to limit the devices and methods described herein, as the discussion may be applied to other similar devices and/or components of devices disclosed herein.

The system 100, accessory devices 102, 200, and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some configurations the sheath can be blended with a liquid crystal polymer (LCP). For example, the mixture can contain up to about 6 percent LCP.

In some configurations, a degree of Magnetic Resonance Imaging (MRI) compatibility is imparted into the system 100 and/or accessory devices 102, 200. For example, the system 100 and/or accessory devices 102, 200 or portions thereof may be made of a material that does not substantially distort the image and create substantial artifacts (i.e., gaps in the image). Certain ferromagnetic materials, for example, may not be suitable because they may create artifacts in an MRI image. The system 100 and/or accessory devices 102, 200 or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

As alluded to above, the tubular and/or elongated components of the system 100 and/or accessory devices 102, 200 may include one or more tubular members that may have slots formed therein. Various configurations of arrangements and configurations of slots are contemplated. For example, in some configurations, at least some, if not all of the slots are disposed at the same or a similar angle with respect to the longitudinal axis of the tubular and/or elongated components of the system 100 and/or accessory devices 102, 200. The slots can be disposed at an angle that is perpendicular, or substantially perpendicular, and/or can be characterized as being disposed in a plane that is normal to the longitudinal axis of the tubular and/or elongated components of the system 100 and/or accessory devices 102, 200. However, in other configurations, the slots can be disposed at an angle that is not perpendicular, and/or can be characterized as being disposed in a plane that is not normal to the longitudinal axis of the tubular components of the system 100 and/or accessory devices 102, 200. Additionally, a group of one or more the slots may be disposed at different angles relative to another group of one or more the slots. The distribution and/or configuration of the slots can also include, to the extent applicable, any of those disclosed in U.S. Pat. No. 7,914,467, the entire disclosure of which is herein incorporated by reference. Some example configurations of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example configurations of etching processes are described in U.S. Pat. No. 5,106,455, the entire disclosure of which is herein incorporated by reference. It should be noted that the methods for manufacturing the system 100 and/or accessory devices 102, 200 may include forming the slots in the tubular or elongated components of the system 100 and/or accessory devices 102, 200 using these or other manufacturing steps.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The invention's scope is, of course, defined in the language in which the appended claims are expressed.

What is claimed is:

1. A steerable medical device comprising:
an elongate tube having a proximal end and a distal end;
a first lumen having a first diameter;
a second lumen having a second diameter smaller than the first diameter;
a steering wire lumen;
a wire filament extending through the steering wire lumen;
a plurality of openings extending from an outer surface of the elongate tube to the steering wire lumen, wherein the wire filament exits and reenters the steering wire lumen through the plurality of openings; and
a handle coupled to the proximal end of the elongate tube and configured to apply tension to the wire filament.

2. The steerable medical device of claim 1, wherein the plurality of openings is axially spaced and circumferentially aligned.

3. The steerable medical device of claim 1, wherein the plurality of openings comprises two or more openings.

4. The steerable medical device of claim 1, wherein the wire filament comprises 304V stainless steel spring temper wire.

5. The steerable medical device of claim 1, wherein a distal end of the wire filament is secured within a hypotube.

6. The steerable medical device of claim 5, wherein the hypotube is crimped at one or more axial locations to secure the wire filament to the hypotube.

7. The steerable medical device of claim 1, wherein the handle comprises:
a body portion; and
an actuating portion movably coupled to the body portion.

8. The steerable medical device of claim 7, wherein the actuating portion is configured to move axially relative to the body portion to apply and release tension to the wire filament.

9. A steerable medical device comprising:
an elongate tube having a proximal end and a distal end;
a first guidewire lumen having a first diameter;
a second guidewire lumen having a second diameter smaller than the first diameter;
a first steering wire lumen;
a second steering wire lumen;
a first wire filament extending through the first steering wire lumen;
a second wire filament extending through the second steering wire lumen;
two or more openings extending from an outer surface of the elongate tube to the first steering wire lumen, wherein the first wire filament exits and reenters the first steering wire lumen through the two or more openings;
two or more openings extending from the outer surface of the elongate tube to the second steering wire lumen, wherein the second wire filament exits and reenters the second steering wire lumen through the two or more openings; and
a handle comprising an actuation member rotatably coupled to a body portion and connected to the first and second wire filaments.

10. The steerable medical device of claim 9, wherein rotation of the actuation member in a first direction applies tension to the first wire filament and releases tension from the second wire filament.

11. The steerable medical device of claim 10, wherein rotation of the actuation member in a second direction opposite the first direction applies tension to the second wire filament and releases tension from the first wire filament.

12. The steerable medical device of claim 9, wherein the first and second wire filaments comprise 304V stainless steel spring temper wire.

13. The steerable medical device of claim 9, wherein distal ends of the first and second wire filaments are secured within a respective first hypotube and second hypotube.

14. The steerable medical device of claim 13, wherein the first hypotube is crimped at one or more locations to secure the first wire filament thereto and the second hypotube is crimped at one or more locations to secure the second wire filament thereto.

15. The steerable medical device of claim 9, wherein the first diameter is 0.035 inches and the second diameter is 0.025 inches.

16. The steerable medical device of claim 9, wherein the first wire filament is releasably secured to the actuation member with a first connection member and the second wire filament is releasably secured to the actuation member with a second connection member.

17. The steerable medical device of claim 16, wherein the first connection member is radially aligned with the second connection member.

18. A steerable medical device comprising:
an elongate tube having a proximal end and a distal end;
a first lumen having a first diameter;
a second lumen having a second diameter smaller than the first diameter;
a steering wire lumen;
a wire filament extending through the steering wire lumen;
a plurality of openings extending from an outer surface of the elongate tube to the steering wire lumen, wherein the plurality of openings defines pivot points; and
a handle coupled to the proximal end of the elongate tube and configured to apply tension to the wire filament.

19. The steerable medical device of claim 18, wherein the wire filament comprises 304V stainless steel spring temper wire.

20. The steerable medical device of claim 18, wherein the handle comprises:
a body portion; and
an actuating portion movably coupled to the body portion.
